(19) United States
(12) Patent Application Publication
(10) Pub. No.: US 2014/0154199 A1
(43) Pub. Date: Jun. 5, 2014

(54) SILICONE-CONTAINING COMPOSITION AND PERSONAL CARE PRODUCTS CONTAINING SAME

(71) Applicant: Momentive Performance Materials, Waterford, NY (US)

(72) Inventors: Anne Dussaud, Tarrytown, NY (US); Bhavna Rana, White Plains, NY (US); Susan Sperring, Pomona, NY (US)

(73) Assignee: Momentive Performance Materials, Waterford, NY (US)

(21) Appl. No.: 13/693,474

(22) Filed: Dec. 4, 2012

(51) Int. Cl. A61K 8/891; A61Q 5/12; A61Q 19/00

(52) U.S. Cl. CPC A61K 8/891; A61Q 19/001; A61Q 5/12
USPC 424/70.12; 424/78.03

(57) ABSTRACT

A silicone composition, especially advantageous for incorporation in a personal care product, includes a silicone resin (a) and a silicone gum (b), the mixture of resin (a) and gum (b) having a softening point of 50° C. or greater and an elastic modulus at ambient temperature of 10⁶ Pa or less.

### FIELD OF THE INVENTION

This invention generally relates to silicone compositions and personal care products containing same. More particularly, the invention relates to silicone compositions containing blends of silicone resin(s) and silicone gum(s) and to personal care products providing shine or gloss, e.g., hair conditioners, hair sprays, hair gels, hair creams, lip glosses, and the like, formulated with such compositions.

### BACKGROUND OF THE INVENTION

The use of linear silicones of high molecular weight has been known for a long time to provide hair conditioning and increase hair luster. However, linear silicones have a tendency to flow and do not provide acceptably stable films on hair fibers. In particular, it is often found that at high dosages of silicone gum, hair fibers tend to stick to each other and the hair takes on a greasy appearance.

It is well known that MQ silicone resin and linear silicone blends form a structured network which is widely used in silicone pressure sensitive adhesives. Such blends are also commonly used for transfer resistance of pigmented formulations in cosmetics, e.g., lipsticks, foundations, and the like. However, in these applications, the blends of silicone resin and linear silicones are tacky and do not provide the clean, non-tacky and smooth finish that would be required of an acceptable hair care product.

### SUMMARY OF THE INVENTION

In accordance with the present invention, there is provided a composition which comprises:

a) silicone resin; and,

b) silicone gum,

the mixture of silicone resin (a) and silicone gum (b) having a softening point of 50° C. or greater and an elastic modulus at ambient temperature of 10⁶ Pa or less.

Surprisingly, it has been found that the foregoing composition forms stable films on hair fibers and significantly reduces the problem of excessive stickiness. When applied to hair, formulations containing the composition of this invention provide high luster while leaving the hair with a smooth feel and with little if any fiber clumping.

### DESCRIPTION OF THE INVENTION

In a preferred embodiment, silicone resin (a) of the composition herein contains one or more M units, represented by the formula R¹₃SiO_{1/3}, and one or more additional units selected from amongst D units, represented by the formula R²₂SiO_{2/2}, T units represented by the formula R³SiO_{3/2} and Q units, represented by the formula SiO_{4/2}, and mixtures thereof, wherein each R¹, R² and R³ is independently a hydroxyl radical or monovalent hydrocarbon radical.

Suitable monovalent hydrocarbon radicals R¹, R² and R³ include acyclic hydrocarbon radicals, alicyclic hydrocarbon radicals and aromatic hydrocarbon radicals. Preferred monovalent hydrocarbon radicals are alkyl radicals, aryl radicals and aralkyl radicals.

As used herein, the expression “acyclic hydrocarbon radical” means a straight or branched chain hydrocarbon radical, preferably containing up 22 carbon atoms, which may be saturated or unsaturated and which may contain one or more hetero atoms, e.g., oxygen, nitrogen, etc., and/or one or more functional groups and/or atoms, e.g., hydroxyl, halo, especially chloro and fluoro, and the like, in substitution of a like number of hydrocarbyl hydrogen atoms.

Suitable monovalent acyclic hydrocarbon radicals include, for example, alkyl, alkenyl, alkynyl, hydroxyalkyl, cyanoalkyl, carboxyalkyl, alkyloxy, oxaalkyl, alkylcarbonyloxaalkylene, carboxamide and haloalkyl, such as, for example, methyl, ethyl, sec-butyl, tert-butyl, octyl, decyl, dodecyl, cetyl, stearyl, ethenyl, propenyl, butynyl, hydroxypropyl, cyanoethyl, butoxy, 2,5,8-trioxadecanyl, carboxymethyl, chloromethyl, trifluoromethyl, and 3,3,3-trifluoropropyl.

As used herein, the expression “alicyclic hydrocarbon radical” means a radical containing one or more saturated hydrocarbon rings, preferably containing from 4 to 12 carbon atoms per ring, which may optionally be substituted on one or more of the rings with one or more alkyl radicals, each preferably containing from 2 to 6 carbon atoms per alkyl radical, halo radicals or other functional groups and which, in the case of a monovalent alicyclic hydrocarbon radical containing two or more rings, may be fused rings. Suitable monovalent alicyclic hydrocarbon radicals include, for example, cyclohexyl and cyclooctyl.

As used herein, the expression “aromatic hydrocarbon radical” means a hydrocarbon radical containing one or more aromatic rings per radical which may optionally, be substituted on the aromatic rings with one or more alkyl radicals, each preferably containing from 2 to 6 carbon atoms per alkyl radical, halo radicals or other functional groups and which, in the case of a monovalent aromatic hydrocarbon radical containing two or more rings, may be fused rings. Suitable monovalent aromatic hydrocarbon radicals include, for example, phenyl, tolyl, 2,4,6-trimethylphenyl, 1,2-isopropylmethylphenyl, 1-pentalenyl, naphthyl, anthryl. As used herein, the term “aralkyl” means an aromatic derivative of an alkyl group, preferably a (C₂-C₆)alkyl group, wherein the alkyl portion of the aromatic derivative may optionally, be interrupted by an oxygen atom such as, for example, phenylethyl, phenylpropyl, 2-(1-naphthyl)ethyl, preferably phenylpropyl, phenyoxypropyl, biphenyloxypropyl, and the like.

In a preferred embodiment, silicone resin (a) contains 30 percent or greater, preferably 40 percent or greater, and more preferably 50 percent or greater, T units with the balance, if any, being made up of one or more Q units, D units, fluorinated groups and mixtures thereof. Thus, e.g., silicone resin (a) can be an MT resin, a fluorinated MT resin, a blend of MT resin with a different silicone resin, e.g., a blend of MT resin and MQ resin, or a blend of fluorinated silicone resins.

In a preferred embodiment, silicone resin (a) is an MT resin in which the ratio of M to T units ranges from 1:1 to 1:7 and the softening point of the resin ranges from 50° to 110° C. Useful silicone resins of this type are disclosed in U.S. 2011/0040062 the entire contents of which are incorporated by reference herein.

Silicone resin (a) is preferably a linear silicone such as polydimethylsiloxane, dimethiconol, amodimethicone, phenyl-modified silicone, silicone block copolymers containing amine groups or quat groups or other charged or uncharged silicone block copolymers, and blends of any of the foregoing. Preferably silicone resin (a) has a number average molecular weight of 500 or greater, preferably 1,000 or greater and up to 1,000,000, and more preferably from 10,000 to 500,000.

In a preferred embodiment, silicone gum (b) of the composition herein is selected from those silicones having a viscosity of from 300 to 200,000,000, and preferably from 750,000 to 2,000,000, centipoise (cps) at 25° C. The viscosity of silicone gum (b) can be readily measured employing known and conventional viscosity measurement apparatus and techniques.

In a preferred embodiment, silicone gum (b) exhibiting the foregoing viscosity characteristic contains M′ units, represented by the formula R⁴₃SiO_{1/2}, and one or more additional units selected from among D′ units, represented by the formula R⁵₂SiO_{2/2}, T′ units, represented by the formula R⁶SiO_{3/2}, and Q′ units, represented by the formula SiO_{4/2}, and mixtures thereof, wherein each R⁴, R⁵ and R⁶ is independently a hydroxyl radical or a monovalent hydrocarbon radical.

Suitable monovalent hydrocarbon radicals R⁴, R⁵ and R⁶ include acyclic hydrocarbon radicals, alicyclic hydrocarbon radicals and aromatic hydrocarbon radicals as defined and exemplified, supra.

Suitable silicone gums (b) are known and are commercially available. For example, the gums can be prepared according to the method disclosed in U.S. Pat. No. 2,814,601, herein incorporated by reference, wherein an appropriate siloxane is reacted with an aqueous acid in a closed system until the viscosity of the siloxane has become essentially constant. The product is then washed free of acid. Specific examples of useful silicone gums (b), all from Momentive Performance Materials Inc., include Silsoft 1215 (dimethiconol gum in cyclodimethicone solvent D5, SE30 (dimethicone gum), Viscasil 60M (polydimethysiloxane gum) and Silsoft AX (alkyl-modified aminosilicone).

When at least a portion of silicon resin(s) (a) and silicone gum(s) (b) possess crosslinkable functionality, e.g., when one or more of R¹, R² and R³ in resin (a) is hydroxyl and one or more of R⁴, R⁵ and R⁶ in gum (b) is hydroxyl (thus presenting silanol reactive groups in the mixture of resin (a) and gum (b), it is within the scope of the invention to provide the mixture of silicone gum(s) (a) and silicone gum(s) (b) or portion thereof as a crosslinked product of resin (a) and gum (b). This embodiment contemplates the use of known and conventional crosslinking procedures, among them being the use of crosslinking agents, catalysts and conditions such as those disclosed in Griswold et al. U.S. Pat. No. 6,890,601, the entire contents of which are incorporated by reference herein.

However, for many products applications including hair care products, it is preferred that all, or nearly all, of the mixtures of silicone resin (a) and silicone gum (b) in accordance with the invention be non-crosslinked. A non-crosslinked mixture of resin (a) and gum (b) will contain little or no crosslinkable functionality, e.g., R¹, R² and R³ of resin (a) and R⁴, R⁵ and R⁶ of gum (b) will contain no more than a few crosslinkable groups, and/or crosslinking conditions will be avoided in the preparation of the mixtures of these silicones and their use in manufacturing the desired personal care product.

The mixture of silicone resin (a) and silicone gum (b) herein must have a softening point of 50° C. or greater, preferably 60° C. or greater and more preferably 70° C. or greater, and an elastic modulus at ambient temperature of 10⁶ Pa or less, preferably 5×10⁵ Pa or less and more preferably 3×10⁵ Pa or less. In general, these characteristics of softening point and elastic modulus can be obtained with mixtures of silicone resin(s) (a) and silicone gum(s) (b) with weight ratios of resin(s) (a) to gum(s) (b) ranging from 0.4 to 6, preferably ranging from 0.5 to 5 and more preferably from 0.7 to 4.

Since the mixtures of silicone resin (a) and silicone gum (b) of this invention have softening points of 50° C. or greater, which is to say, they are solids at ambient temperature, it may be advantageous for formulating a particular personal care product to dilute the mixture of silicone resin(s) (a) and silicone gum(s) (b) with one or more volatile organic solvents (c), e.g., an organosilicon-containing solvent such a ethyl trisiloxane, octyl trisiloxane, cyclodimethicone, and the like, and/or one or more other type organic solvents such as the volatile paraffinic solvents and the aromatic hydrocarbon solvents. Examples of such solvents include the C₅-C₁₂ acyclic and cyclic alkanes, e.g., the straight chain and isomeric pentanes, hexanes, heptanes, octanes, nonanes, decanes, undecanes, dodecanes, etc., and their cyclic analogs, and aromatic solvents as, for example, exemplified by benzene, toulene, the xylenes, mesitylene, and the like. Where utilized, volatile organic solvent(s) (c) can generally be combined with mixtures of resin(s) (a) and gum(s) (b) in a weight ratio of solvent to resin/gum mixture from 200 to 0.1, preferably from 100 to 1 and more preferably from 60 to 5.

In a preferred embodiment, an organic solvent solution of resin (a) and gum (b) can be formulated as an aqueous or non-aqueous spray, an aqueous or non-aqueous foam or mousse, a water-in-oil (w/o) emulsion or oil-in-water (o/w) emulsion employing procedures well known in the art for providing hair care products.

The personal care formulations of the invention can also contain one or more other ingredients known for use in such products in known and conventional amounts such as humectants (panthenol, butylene glycol, sorbitol, glycerin, other polyols), amino acids, natural moisturizing factors (sodium PCA), nonionic waxes (fatty alcohols, ethoxylated waxes, glycerol stearate, bee waxes, paraffin waxes etc.), esters, triglyceride oils (olive oil, jojoba oil, sunflower oil, coconut oil, argan oil, grapeseed oil etc.), natural butters (shea butter, cocoa butter), emulsifiers (silicone emulsifiers, silicone polyether copolymers, organic emulsifiers), anionic or amphoteric surfactants (cocobetaine, SLES, isothionate, sugar surfactants), spreading agents such as silicone superspreaders, solid particulates, pigments, minerals (talc, micas, iron oxides, boron nitride, titanium oxide, clays), permanent and semi-permanent hair dyes, fragrances, actives such as plant extracts, polyphenols, polysaccharides (chitosan), proteins (keratin, silk protein, wheat proteins), lipids, sterols, antidandruff actives, salicylic acid, glycolic acid, hair growth actives, anti-aging actives (retinol, alpha-hydroxy acids), niacinamide, reducing agents (thioglycolates, cysteamine), sulfites, oxidizing agents (hydrogen peroxide, bromates), relaxers (sodium hydroxides, guanidines), crosslinking agents (aldehydes, epoxy containing compounds, silanes, enzymes), styling polymers (PVP, acrylate copolymers), thickening polymers (acrylates, polyacrylamide, cellulose, starch, polysaccharide gums, pectins, etc.), deposition aid polymers (cationic guars, cationic cellulose, merquats), preservatives, biocides (phenoxyethanol, potassium sorbate, benzoic acid, sorbic acids, etc.), antioxidants (vitamin E), UVA UVB sunscreens, sunless tanning agents (dihydroxyacetone), and so forth.

### COMPARATIVE EXAMPLES 1-6; EXAMPLES 1-7

### (1) Silicone Resins (a) and Silicone Gums (b)

| | | |
|---|---|---|
| Silicone Resin | Silform Flex | MT Resin |
| Silicone Resin | SR1000 | MQ Resin |
| Linear Silicone in | Silsoft 1215 | Dimethiconol Gum |
| Volatile Silicone D5 | | |
| Linear Silicone | SE30 | Dimethicone Gum |
| Linear Silicone | Viscasil 60M | Polydimethylsiloxane Gum |
| Linear Silicone | Silsoft AX | Alkyl-modified Aminosilicone |

Silform Flex MT resin and SR1000 MQ resin are available from Momentive Performance Materials Inc. Silsoft 1215 contains 15% by weight of dimethiconol gum in silicone solvent D5 and is available from Momentive Performance Materials Inc. SE30 is a dimethicone gum also available from Momentive Performance Materials Inc. Viscasil 60 M is a polydimethylsiloxane gum having a viscosity of about 60,000 cps and is available from Momentive Performance Materials Inc.

### (2) Test Methods

a. Softening Point and Elastic Modulus

Mixtures of resin (a) and gum (b) with different weight ratios of resin (a) to gum (b) were dissolved in cyclodimethicone solvent D5, placed in a flat aluminium pan and dried at 90° C. until the D5 had completely evaporated. Elastic modulus (G′) and storage modulus (G″) were measured as functions of the temperature in the range of from −150° C. to 150° C. employing Dynamic Mechanical Analysis of TA Instrument (New Castle, Del.), at a frequency of about 1 Hz. The temperature where the maximum of tan delta (ratio of G″/G′) was observed was defined as the softening temperature. This method is a well established method for characterizing silicone pressure sensitive adhesives.

b. Film Tack

60 micron films were produced in aluminium pans by drying mixtures of resin (a) and gum (b) in solvent D5. After evaporation of the D5, maximum tack forces were measured with a Stable Micro Systems Texture Analyzer (Surrey, U.K.) at a load of 100 g and a contact time of 1 s. Hair care products exhibiting a level of film tack not greater than 50 g are generally considered to perform acceptably well in this regard.

c. Hair Gloss (or Luster)

Hair luster was measured with a Murakami GoniophotoMeter (Tokyo, Japan), at an angle of incidence of 30°. Gloss factor was obtained using the total reflectance values and the formula F_{g}=(Lₘₐₓ−D)/D where Lₘₐₓ is the maximum reflectance and D is the reflectance value obtained at 0° viewing angle (diffusive reflectance). Hair luster is considered to be significantly increased when the gloss factor is higher than 1.6.

d. Tress Volume

Fiber clumping produced a significant volume reduction of the tress. Volume reduction was measured by image analysis after the hair tress was stored 1 hour in a 90% RH humidity chamber. The tress volume was measured by counting the number of pixels of the tress area (A). A volume reduction factor was obtained using the formula Rᵥ=100*(Aₒ−A)/(Aₒ−Aₘᵢₙ) where Aₒ was the value of the untreated tress area (very frizzy tress) and Aₘᵢₙ was the minimum tress area (ironed tress). If the volume reduction is excessive (Rᵥ>70%), the hair will appear clumpy. If the volume reduction is too low (Rᵥ<30%), the hair will appear frizzy. With a volume reduction between 40% and 60%, treated hair tress will not appear frizzy and the hair fibers will not form clumps, thus allowing the hair to flow freely.

e. Hair Smoothness

Hair coefficient of friction μ was measured on a CSM tribometer (Needham, Ma), on a taut flat tress, with a flat stainless steel probe. Hair was considered smooth when μ<0.12.

### (3) Hair Treatment Formulations

Examples of mixtures of resin (a) and gum (b) blends and the results of treating hair with the mixtures are presented in Tables 1A and 1B below. The mixtures of resin (a) and gum (b) were diluted in cyclodimethicone D5 to provide a 2% by weight solids solution. A single bleached hair tress was immersed in each test solution for 1 min. Excess liquid was squeezed out and the tress was thoroughly dried to remove solvent D5 using a blow drier, the tress thereafter being placed in an oven at 45° C. overnight. By this procedure, approximately the same amount of silicone mixture was delivered to each hair tress sample. Tress measurements were taken after equilibration in a 50% humidity chamber.

In Tables 1A and 1B, Comparative Example 1 is an untreated hair tress having low luster, low tack and a rough finish. In Comparative Example 2, the tress treated only with silicone gum had a very high luster but exhibited excessive clumping of its fibers resulting in the tress appearing greasy. The mixtures of resin (a) and gum (b) of Comparative Examples 3 and 4, both of which had very low softening points, produced high tack films and excessive hair fiber clumping. The mixtures of resin (a) and gum (b) of Comparative Examples 5 and 6, both of which had a high elastic modulus at room temperature, resulted in a hair with low luster and a rough finish.

In contrast to the compositions of Comparative Examples 1-6 and as shown in Table 1B, the hair tresses of Examples 1-7 which are illustrative of the invention produced desirable properties of high gloss, low friction (smooth feel) and moderate volume reduction, i.e., no appreciable clumping. With a volume reduction between 40% and 60%, the hair tresses treated with the compositions of Examples 1-7 did not appear frizzy and the hair fibers avoided forming clumps as shown by the hair being able to flow freely. The silicon mixtures of Examples 1-7 therefore fulfilled all major criteria for a well-formulated and functioning hair care product whereas Comparative Examples 1-6 failed to meet even one of these criteria.

TABLE 1A
Mixtures of Resin (a) and Gum (b)

| | | | | | Weight Ratio |
|---|---|---|---|---|---|
| | % | % | % T unit in | | of Resin (a) to |
| Example | MQ | MT | Resin | Gum (b) | Gum (b) |
| Comp Ex. 1 | | | | | |
| Comp Ex. 2 | 0 | 0 | | Silsoft 1215 | |
| Comp Ex. 3 | 100 | | 0 | Silsoft 1215 | 0.5 |
| Comp Ex. 4 | 100 | | 0 | Silsoft 1215 | 1.3 |
| Comp Ex. 5 | 100 | | 0 | Silsoft 1215 | 3.1 |
| Comp Ex. 6 | 100 | | 0 | Silsoft 1215 | 4.7 |
| Ex. 1 | | 100 | 85 | Silsoft 1215 | 0.8 |
| Ex. 2 | | 100 | 85 | Silsoft 1215 | 1.3 |
| Ex. 3 | | 100 | 85 | Silsoft 1215 | 2.1 |
| Ex. 4 | | 100 | 85 | Silsoft 1215 | 3.1 |
| Ex. 5 | | 100 | 85 | Silsoft 1215 | 4.7 |
| Ex. 6 | 20 | 80 | 68 | Silsoft 1215 | 1.3 |
| Ex. 7 | 33 | 67 | 57 | Silsoft 1215 | 1.3 |

TABLE 1B
Results of Hair Treatment

| | Softening | | | | | | |
|---|---|---|---|---|---|---|---|
| | Point of | | | | | Hair | |
| | Mixture, | G′(Pa) at | Tack | Gloss | Volume | Coefficient | |
| Example | (° C.) | 25° C. | (g) | Factor | Reduction % | of Friction | Result(s) |
| Comp Ex. 1 | | | 0 | 1.23 | 0 | 0.147 | low shine, frizzy hair, poor feel |
| Comp Ex. 2 | −50 | 3.6 × 10⁴ | 19 | 2.46 | 82 | 0.084 | Clumping |
| Comp Ex. 3 | 17 | 1.7 × 10⁵ | 113 | 2.57 | 79 | 0.101 | Clumping |
| Comp Ex. 4 | 43 | 3.3 × 10⁵ | 161 | 2.16 | 86 | 0.100 | Clumping |
| Comp Ex. 5 | 82 | 3.4 × 10⁶ | 0 | 1.53 | 50 | 0.146 | low shine, draggy |
| Comp Ex. 6 | 82 | 2.5 × 10⁶ | 0 | 1.48 | — | 0.155 | low shine, draggy |
| Ex. 1 | 108 | 4.5 × 10⁴ | 30 | 2.3 | 56 | 0.093 | high shine, no clumping, smooth |
| Ex. 2 | 106 | 4.5 × 10⁴ | 25 | 2.48 | 55 | 0.080 | high shine, no clumping, smooth |
| Ex. 3 | 94 | 1.0 × 10⁵ | 7 | 2.71 | 49 | 0.097 | high shine, no clumping, smooth |
| Ex. 4 | 90 | 1.8 × 10⁵ | 3 | 1.93 | 45 | 0.091 | high shine, no clumping, smooth |
| Ex. 5 | 85 | 2.1 × 10⁵ | 3 | 2.51 | — | 0.094 | high shine, no clumping, smooth |
| Ex. 6 | 92 | 7.5 × 10⁴ | 37 | 1.83 | — | | high shine, no clumping, smooth |
| Ex. 7 | 93 | 4.9 × 10⁴ | 22 | 1.62 | — | | high shine, no clumping, smooth |

### COMPARATIVE EXAMPLE 7; EXAMPLE 8

It is an essential requirement of a hair care product containing a mixture of silicone resin (a) and silicone gum (b) that it be essentially devoid of cationic surfactant. This requirement is demonstrated by the comparison presented below in Table 2.

TABLE 2
Results of Hair Treatment With Mixtures of Resin (a)
and Gum (b) With and Without Cationic Surfactant

| | Comparative | |
|---|---|---|
| Hair Treatment | Example 7 | Example 8 |
| MT Resin | 10 | 10 |
| Gum SE30 | 5 | 5 |
| Cyclodimethicone D5 | 30 | 85 |
| cetrimonium chloride | 2 | 0 |
| (cationic surfactant) | | |
| Water | 53 | 0 |
| ratio Resin (a)/Gum (b) | 2 | 2 |
| % T Units in Resin (a) | 85 | 85 |
| Gloss Factor | 1.5 | 2.14 |
| Tress Appearance | Clumping, | High Shine, |
| | Greasy | No Clumping |

As shown in Table 2, the tress treated with a composition of the invention (Example 8) containing no cationic surfactant exhibited a high shine with no fiber clumping thus giving the hair a clean appearance with its fibers moving freely. In contrast, the hair treated with the composition containing cationic surfactant (Comparative Example 7) appeared clumpy and greasy.

### EXAMPLES 9-14

Table 3 below sets forth the composition of 0/W emulsions suitable for the formulation of hair care products in accordance with the invention. In accordance with the invention, each of the illustrated compositions contains a mixture of silicone rein (a) and silicone gum (b) and no cationic surfactant.

TABLE 3
O/W emulsion examples

| Ingredient | | | | Example | Example | Example | Example | Example |
|---|---|---|---|---|---|---|---|---|
| Tradename | INCI* | Description | Example 9 | 10 | 11 | 12 | 13 | 14 |
| AMP-95 | aminomethyl propanol | | 0.56 | | | | | 0.7 |
| Aculyn 180 | acrylates/hydroxyesters | Anionic polymer | 2 | | | | | |
| | acrylates copolymer | | | | | | | |
| Aculyn88 | acrylates/steareth 20 | Anionic polymer | 5 | | | | | |
| | methacrylate copolymer | | | | | | | |
| Cellosize polymer | hydroxyelthyl cellulose | nonionic polymer | | | | | | |
| peg-10 | | | | | | | | |
| Sepigel 305 | | Anionic polymer | | 5 | | | | |
| Aristof lex AVC | | Anionic polymer | | | 2 | | | |
| Carbopol 980 | | Anionic polymer | | | | | | 0.9 |
| Fixate G-100 | AMP-acrylates/allyl | Anionic polymer | | | | | | 2.6 |
| | meth acrylates | styling polymer | | | | | | |
| | copolymers (26%) | | | | | | | |
| Ultrez 20 | acrylates/C10-C30 allky I | Anionic polymer | | | | 0.2 | | |
| | acrylate copolymer | | | | | | | |
| Carbopol aqua SF- | | Anionic polymer | | | | | 1 | |
| 1 (30%) | | | | | | | | |
| | glyceryl stearate and | nonionic | | | | 2 | 6 | |
| | PEG-100stearate | surfactant | | | | | | |
| | cetearyl alcohol | fatty alcohol wax | | | | 1 | 3 | |
| | sodium hydroxide (18%) | | | | | 0.2 | 0.2 | |
| | panthenol | humectant | | | | 0.5 | 0.5 | |
| | disodium EDTA | | | | | 0.05 | 0.05 | |
| Silicone MT Resin | | Silicone resin | 5 | 5 | 5 | 5 | 5 | 5 |
| Silicon Gum SE30 | | Silicone gum | 2.5 | 2.5 | 2.5 | 2.5 | 2.5 | 2.5 |
| Cyclodimethicone | | Volatile silicone | 7 | 7 | | | | 7 |
| D5 | | | | | | | | |
| isododecane | | Volatile solvent | | | | | 7 | |
| Silsoft ETS | | volatile silicone | | 7 | 7 | 7 | | |
| water | | | q.s 100 | q.s 100 | q.s 100 | q.s 100 | q.s 100 | q.s 100 |

*INC = International Nomenclature of Cosmetic Ingredients

### EXAMPLES 15 AND 16

Table 4 below sets forth the composition of WO emulsions suitable for formation of hair care products in accordance with the invention.

TABLE 4
W/O emulsion example

| | | Example 16, | Example 17, |
|---|---|---|---|
| Ingredient | Description | Wt % | Wt % |
| Silform 60A | Silicone polyether | 2 | 2 |
| | copolymer | | |
| Isododecane | Volatile solvent | 10 | 10 |
| Silsoft ETS | Volatile Silicone | 10 | 10 |
| MT resin | Silicone Resin | 5 | 5 |
| Silicone gum SE30 | Silicone Gum | 2.5 | — |
| Silsoft AX | Alkyl-modified | — | 1.6 |
| | aminosilicone | | |
| NaCl | | 0.8 | 0.8 |
| Butylene glycol | Humectant | 3 | 3 |
| Water | | 66.7 | 66.7 |

### EXAMPLE 17

Table 5 sets forth the formulation of a lip gloss product in accordance with the invention.

TABLE 5
Lip Gloss Formulation

| Ingredient | Wt % |
|---|---|
| MT resin | 30 |
| Viscasil 60M gum | 15 |
| Cyclodimethicone | 54.8 |
| Fragrance | 0.2 |

While the invention has been described with reference to particular embodiments, those skilled in the art will understand that various changes may be made and equivalents may be substituted for elements thereof without departing from the scope of the invention. It is intended that the invention not be limited to the particular embodiments disclosed but that it include all embodiments falling within the scope of the appended claims.

1. A composition which comprises:
a) silicone resin; and,
b) silicone gum,
the mixture of silicone resin (a) and silicone gum (b) having a softening point greater than 50° C. and an elastic modulus at ambient temperature of less than 10⁶ Pa.

2. The composition of claim 1 wherein silicone resin (a) contains one or more M units of the formula R¹₃SiO_{1/2} and one or more additional units selected from amongst D units of the formula R²₂SiO_{2/2} T units of the formula R³SiO_{3/2} and Q units of formula SiO_{4/2}, and mixtures thereof, wherein each R¹, R² and R³ is independently a hydroxyl radical or monovalent hydrocarbon radical and silicone gum (b) possesses a viscosity of from 300 to 200,000,000 centipoise at 25° C.

3. The composition of claim 1 wherein silicone gum (b) contains one or more M′ units of the formula R⁴₃SiO_{1/2} and one or more additional units selected from amongst D′ units of the formula R⁵₂SiO_{2/2}, T′ units of the formula R⁶SiO_{3/2} and Q′ units of the formula SiO_{4/2}, and mixtures thereof, wherein each R⁴, R⁵ and R⁶ is independently a hydroxyl radical or a monovalent hydrocarbon radical.

4. The composition of claim 2 wherein silicone resin (a) contains 30% or greater T units with the balance, if any, being made up of one or more Q units, D units, fluorinated groups or mixture thereof.

5. The composition of claim 1 wherein silicone resin (a) is at least one of MT resin, fluorinated MT resin, blend of MT resin and at least one other silicone resin and fluorinated silicone resin and silicon gum (b) is at least one of dimethiconol gum, dimethicone gum and polydimethysilicone gum.

6. The composition of claim 1 wherein the weight ratio of silicone resin (a) to silicone gum (b) ranges from 0.4 to 6.

7. The composition of claim 1 which is essentially cationic surfactant-free.

8. The composition of claim 1 in which the mixture of silicone resin (a) and silicone gum (b) is non-crosslinked.

9. The composition of claim 7 in which the mixture of silicone resin (a) and silicone gum (b) is non-crosslinked.

10. The composition of claim 1 further comprising a volatile organic solvent for the mixture of silicone resin (a) and silicone gum (b).

11. A personal care product which comprises:
a) silicone resin;
b) silicone gum; and,
c) volatile organic solvent for the mixture of silicone resin (a) and silicone gum (b),
the mixture of silicone resin (a) and silicone gum (b) having a softening point of 50° C. or greater and a modulus at ambient temperature of 10⁶ Pa or less.

12. The personal care product of claim 11 wherein silicone resin (a) contains one or more M units of the formula R¹₃SiO_{1/3} and one or more additional units selected from amongst D units of the formula R²₂SiO_{2/2}, T units of the formula R³SiO_{3/2}, Q units of formula SiO_{4/2}, and mixtures thereof, wherein each R¹, R² and R³ is independently a hydroxyl radical or monovalent hydrocarbon radical, and silicone gum (b) possesses a viscosity of from 5,000 to 200,000,000 centipoise at 25° C.

13. The personal care product of claim 11 wherein silicone gum (b) contains one or more M′ units of the formula R⁴₃SiO_{1/2} and one or more additional units selected from amongst D′ units of the formula R⁵₂SiO_{2/2}, T′ units of the formula R⁶SiO_{3/2} and Q′ units of the formula SiO_{4/2}, and mixtures thereof, wherein each R⁴, R⁵ and R⁶ is independently a hydroxyl radical or a monovalent hydrocarbon radical.

14. The personal care product of claim 12 wherein silicone resin (a) contains at least 30% T units with the balance, if any, being made up of Q units, D units, fluorinated groups or mixtures thereof.

15. The personal care product of claim 11 wherein silicone resin (a) is at lest one of MT resin, fluorinated MT resin, blend of MT resin and at least one other silicone resin, and fluorinated silicone resin, and silicone gum (b) is at least one of dimethiconol gum, dimethicone gum and polydimethysilicone gum.

16. The personal care product of claim 11 wherein the weight ratio of silicone resin (a) to silicone gum (b) ranges from 0.4 to 6.

17. The composition of claim 11 wherein the personal care product is an essentially cationic surfactant-free hair care product in which the mixture of silicone resin (a) and silicone gum (b) is non-crosslinked and is dissolved in volatile organic solvent.

18. The personal care product of claim 17 wherein hair treated with the hair care product satisfies at least two of the following criteria: a film tack of not greater than 50 g, a hair gloss factor of at least 1.7, a tress volume reduction value of from 40 to 70% and a hair coefficient of friction of less than 0.12.

19. The composition of claim 1 wherein the personal cared product is a lip gloss.

20. A method of treating hair which comprises treating hair with the hair care composition of claim 17, the treated hair satisfying at least two of the following criteria: a film tack of not greater than 50 g, a hair gloss factor of at least 1.6, a tress volume reduction value of from 40 to 70% and a hair coefficient of friction of less than 0.12.
